(19) United States
(12) Patent Application Publication
(10) Pub. No.: US 2009/0312722 A1
(43) Pub. Date: Dec. 17, 2009

(54) INJECTION FLUID LEAKAGE COLLECTION SYSTEM AND METHOD

(76) Inventors: Philippe E. Laurent, Oulins (FR); Ronald J. Pettis, Cary, NC (US); William A. Easterbrook, III, Westwood, NJ (US); Julie Berube, Ringwood, NJ (US)

Correspondence Address: David W. Highet, VP & Chief IP Counsel;Becton, Dickinson and Company, 1 Becton Drive, MC 110, Franklin Lakes, NJ, 07417-1880, US

(21) Appl. No.: 11/997,663

(22) PCT Filed: Aug. 3, 2006
(86) PCT No.: PCT/US06/30574

§ 371 Date: Sep. 9, 2008

Related U.S. Application Data
(60) Provisional application No. 60/705,509, filed on Aug. 4, 2005.

(51) Int. Cl. A61F 13/15

(52) U.S. Cl. 604/289

(57) ABSTRACT

A system and method for collection of fluids, as may be used in evaluation of drag dose delivery completeness after parenteral injection by measuring fluid volume leakage from the injection site is provided. The system and method optionally separate the collection and measurement steps, which make the system and method easy to use in multi-site clinical trials, and for batch weighing operations.

### FIELD OF THE INVENTION

Aspects of the present invention are directed to a method and device for measurement of post-injection leakage of a fluid. Such an invention may be particularly useful in connection with intradermal delivery to a patient. Other uses may be fluid collection with delayed parameter measurement. Certain aspects of the invention are directed to a system and kit for containing and measuring the leakage a substance from a patient.

### BACKGROUND OF THE INVENTION

Intradermal delivery methods and devices using a cannula are effective for many applications. However, in certain circumstances, the leakage (potentially induced by such shallow delivery) has prompted the development of leakage testing methods. Recently, a number of intradermal devices employing microneedles have been designed. The microneedles have a length selected to penetrate the skin to a depth where a drug or pharmaceutical agent can be delivered to a patient. In some situations, these microneedles have allowed smaller and smaller doses to be delivered to the patient. The assessment of the minimal leakage that occurs from such new devices has prompted the need for the development of a highly precise and accurate leakage characterization system.

A variety of references have emphasized the importance and or attempted to characterize the potential leakage fault of certain types of intradermal injections such as: Belshe R B, Newman F K, Cannon J, Duane C, Treanor J, Van Hoecke C et al. Serum antibody responses after intradermal vaccination against influenza. N Eng J Med 2004; 351:22-31; Kenney R, Frech S A, Muenz L R, Villar C P, Glenne G M. Dose sparing with intradermal injection of influenza vaccine. N Eng J Med 2004; 351; Flynn P M, Shenep J L, Mao L. Crawford R, Williams B F, Williams B G. Influence of needle gauge in Mantoux skin testing. Chest 1994; 106:1463-1465; Bremseth D L, Pass F. Delivery of insulin by jet injection: recent observations. Diabet Tech Ther 2001; 3:225-232; Hanas R, Lytzen L, Lugvigsson J. Thinner needles do not influence injection pain, insulin leakage or bleeding in children and adolescents with type 1 diabetes. Ped Diabet. 2000; 1:142-149; Van Doom L, Alberda A, Lytzeen L. Insulin leakage and pain perception: comparison of 6 mm and 12 mm Novofine® needles in patients with type I and type II diabetes. Diabet Med 1998; 15 (suppl 1): S50 and; Stewart N L, Darlow B A. Insulin loss at the injection site in children with type 1 diabetes mellitus. Diabet Med 1994; 11:802-805.

Needle based parenteral injections devices targeting skin dermis or shallow hypodermis delivering fluid volume in the range of 100 to 200 μL have been developed for drug and vaccine delivery. Precise and accurate measurement of the completeness of the injection and the consistency of the effective injected fluid volume in body is one of the most critical criteria to evaluate the effectiveness of the new injection technique and devices. In some situations, the fluid to be delivered to the patient is not fully delivered to the target tissue, and as such, it is sometimes called a “wet injection,” as the delivery site is literally wet with the fluid of injection. Wet injections due to fluid leakage from the device, the injection site, or a combination of both, has been reported as a possible root cause of dose delivery variability with both needle and needle free injection systems. The prior methods and devices for the measurement of leakage from intradermal administration of substances have exhibited limited success. Furthermore, it may be desirable to separate the collection step and delay the measurement step until the measurement can conveniently be made. Accordingly, a continuing need exists for an improved device and method for the precise and accurate measurement of leakage of various drugs and other substances from the body with a validated fluid leakage collection and volume measurement method, which is easy to use in clinical trials for injection performance evaluations.

### SUMMARY OF THE INVENTION

An aspect of the present invention is directed to a method and device for the collection and measurement of a substance, which effuses from the skin of a patient after an injection, such as, an intradermal injection. More particularly, aspects of the invention are directed to methods and devices for measuring a quantity of a pharmaceutical agent, such as a drug or vaccine, which has leaked from the skin. Other aspects of the invention are further directed to systems, kits and methods for measurement of the component of a dose, which was not delivered through the skin of a patient.

In one aspect of the invention, a method is provided for the evaluation of drug dose delivery completeness after parenteral injection by measuring fluid volume leakage from the injection site. The method is easy to use in multi-site clinical trials and allows precise and accurate fluid leakage collection, storage and measurement. A wicking spear is combined with a gravimetric method and delayed measurement, thus, a measurement method is provided for fluid volumes ranging from 0 to 100 μL and a measurement error below 1 μL due to intermediate storage of the sample after fluid leakage collection for up to 12 days (at room temperature). The practitioner is able to collect the fluid effluent and delay measurement in order to measure each sample in a batch type operation. This allows accurate and precise fluid leakage measurement at a different time/location/operator than the injection. The volume detection threshold is below 2 μL, the method capability at 3a ranges from 80.49% for 2 μL to 97.74% for 25 μL.

The objects and advantages of the invention are further attained by providing a leakage testing device comprising a wicking portion having at least a portion made of a fibrous material with sufficient porosity to allow filling thereof by capillary action and when the fibrous material is filled with the effluent. When capillary action is utilized for the filling action of the fibrous material, methods are provided herein for sizing the fibrous material appropriately.

The device and method of the present invention are suitable for use in measuring the effectivity of the administration of various substances, including pharmaceutical agents, to a patient, and particularly to a human patient. As the term is used herein, a pharmaceutical agent includes a substance having biological activity that can be delivered through the body membranes and surfaces, particularly the skin. Examples of pharmaceutical agents include antibiotics, antiviral agents, analgesics, diagnostics, anesthetics, anorexics, antiarthritics, antidepressants, antihistamines, anti-inflammatory agents, antineoplastic agents, vaccines, including DNA vaccines, and the like. Other substances that can be measured and delivered intradermally to a patient include proteins, peptides and fragments thereof. The proteins and peptides can be naturally occurring, synthesized or recombinantly produced.

The measurement device of the present invention is constructed for collection of fluids to attain the desired precision and accuracy of the drug delivery system. The desired precision for both the measurement device and the drug delivery device is determined by the therapeutic index of the substance being delivered and the desired rate of absorption by the body.

The objects, advantages and other salient features of the invention will become apparent from the following detailed description which, taken in conjunction with the annexed drawings, discloses preferred embodiments of the invention.

### BRIEF DESCRIPTION OF THE DRAWINGS

The foregoing and other features and advantages of the invention will be apparent from the following, more particular description of a preferred embodiment of the invention, as illustrated in the accompanying drawings wherein like reference numbers generally indicate identical, functionally similar, and/or structurally similar elements.

FIGS. 1a-1b depict Boxplots of percentage recovery comparing capillary and wicking spear methods which are exemplary embodiments of a measurement device according to an embodiment of the present invention;

FIG. 2 depicts a scatter-plot of the volume of collected fluid vs. volume distributed with the pipette.

FIG. 3A-B depicts an exemplary embodiment of a post injection fluid leakage measurement kit.

FIG. 4A-D depict an exemplary embodiment of a post injection fluid leakage measurement kit using wicking spear method.

FIG. 4A: depicts an exemplary embodiment of a wicking spear;

FIG. 4B depicts an exemplary embodiment of a wicking spear in an sealable tube;

FIG. 4C: depicts an exemplary embodiment of a wicking spear collecting fluid leakage on skin surface;

FIG. 4D: depicts an exemplary embodiment of a sample collection box for kit shipment before and after fluid leakage collection.

### DETAILED DESCRIPTION OF THE PREFERRED EMBODIMENTS

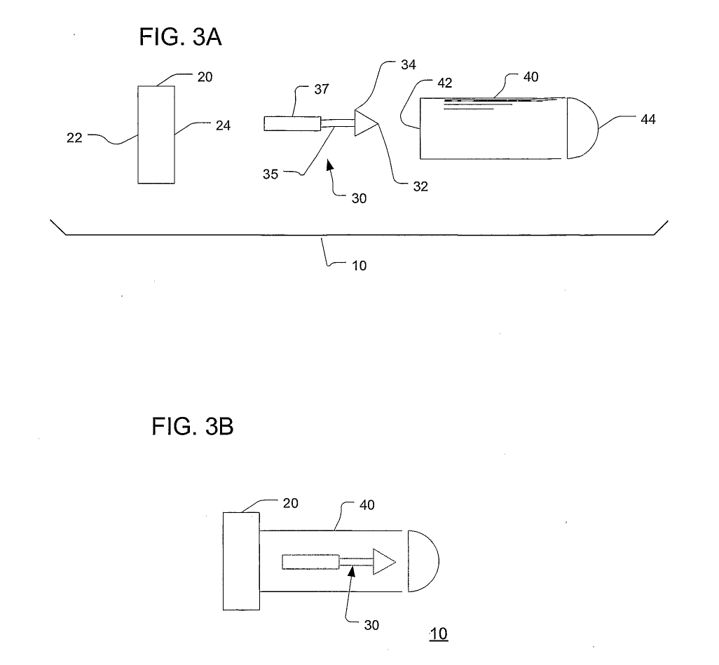

As can be seen from FIG. 3A-3B, an embodiment displaying aspects of a fluid collection kit according to one implementation of the present invention, which is designated generally by the reference numeral 10, comprises a containment tube 40 and cap 20 and a fluid collection device 30. Cap 20 is associated with containment tube 40 to contain the collected fluid volume residing on fluid collection device 30. Cap 20 has both distal end 24 which as an engagement means to proximal end 42 of tube 40. The engagement means provides for closure of open proximal end 42 of tube 40. The engagement may be a variety of means such as a stopper, threads, a slip fit, or the like. Fluid collection device comprises a generally elongated body 35. The fluid collection device 30 also contains a porous portion 34 with a distal end 32. Porous portion 34 is sized to have porosity, which will allow collection of the maximum amount of fluid leakage anticipated to be collected. The device also has a gripping portion 37 disposed on the body 35 of the fluid collection device 30. Alternatively, collection device 30 may be constructed entirely of the material of the porous portion. Fluid collection device 30 has a specific mass, which is determined prior to use for collection of fluid. The mass is recorded prior to use. The mass may be printed on a label affixed to the outside portion of tube 40. Alternatively, the mass of collection device 30 is printed directly on collection device 30. The information (mass, code, etc.) contained on collection device 30 may be in the form of alpha-numeric characters, bar codes, or other information encoding methods well known to one skilled in the art.

In order to use the device, a mass measurement of wicking device 30 is taken and recorded. Alternatively, the entire kit 10 is weighed. Device 30 is then placed into tube 40 and sealed with cap 20. Subsequently a practitioner gathers the materials required for injection (syringe, pen, etc.) and for fluid collection (at least one fluid collection kit 10). Immediately before or after an injection is made, cap 20 is removed from tube 40 and device 30 is removed from tube 40. A practitioner, while holding device 30 by handle 37, places distal tip 32 of device 30 proximate to the injection site and subsequently porous portion 34 collects substantially all the fluid effluent from the injection site. The practitioner then places device 30 back into tube 40 and seals tube 40 with cap 20. At the time of the final mass measurement, cap 20 is removed from tube 40, device 30 is removed from tube 40, and the mass is determined. Alternatively, the entire kit 10 is weighed, in which case removal of device 30 is not required as it already contains the mass of interest. The first mass measurement is subtracted from the second mass measurement and a fluid leakage mass is determine. From that measurement, a fluid leakage volume can be calculated, when the density of the effluent is known.

In the case of plural fluid collection kits 10, the use of a rack 50, with a plurality of receiving openings 55 may be utilized. Rack 50 ensures proper confinement and containment of fluid collection kits 10, which may be deemed as hazardous materials based on the effluent collected.

The use of tube 40 and cap 20 as a storage container between time of the mass measurement of collection device 30 and the use of collection device 30 to collect fluid minimizes errors in later mass measurements due to material loss/gain to the collection device 30 by a variety of factors including but not limited to: addition of residual oils, water loss/gain, and fiber loss/gain. Furthermore, the use of tube 40 and cap 20 as a storage container between time of the fluid collection by collection device 30 and the final mass measurement of collection device 30 minimizes errors in mass measurements due to material loss/gain to the collection device 30.

The capillary tube method uses length measure of collected fluid column height and correlates the length measured to a collected volume result using a ratio calculation of length measure taken from the average of ten-to-5 L calibration mark measurements. The wicking spear method, so called gravimetric method, uses the weight difference between a dry wicking spear before and after being used to collect the leaked fluid to correlate a collected volume result in μL.

The formulae are as follows:

Collected Vol. Cap(μL)=[Fluid column Height/Length(mm)÷Average(n=10)tip-to-5 μL Length(mm)]×5 μL.

Collected Vol. Wicking spear(μL)=[Spear weight post collection(g)−Spear weight pre collection(g)]×1000 μL/g

A mathematic approximation for filling of the porous material to fill may be modeled by Washburn's equation which describes capillary flow in porous materials.

Washburn's equation is:

$${L}^{2} = \frac{γ Dt}{4 η}$$

where t is the time for a liquid of viscosity η and surface tension γ to penetrate a distance L into a fully wettable, porous material whose average pore diameter is D. From this equation, the wick may be selected for the desired fluid capture parameters (time, and volume), by selecting the parameters (pore density/diameter, wick size) of the wick appropriately, using known values for the fluid viscosity and surface tension. The radial dimension L may be approximated by the average distance from the external surface of the wick to the geometric center (or centroid) of the wick. Furthermore, the center of mass would coincide with the centroid of a wick of uniform density. For example, for a spherical wick shape the dimension L would be precisely equal to the radius of the sphere, having its centroid at the center of the sphere. For other three-dimensional shapes, a centroid may be calculated and a dimension L may be approximated by determining the average distance from the external surface of the wick to the geometric center (or centroid) wick. Alternatively, for a wick which is triangular, dimension L may be determined by using the average distance from the contacting tip to the centroid of the wick, as the fluid is entering the wick at the contacting tip point of the triangular section.

### Material and Methods

In each of the examples, the objective was to evaluate each of the two different fluid leakage collection methods to meet acceptance criteria according to fluid volume range. The secondary objective was to use this data to also comparatively evaluate the two methods to determine if they are statistically different taking into account the operator effect. The tertiary objective was to validate the wicking spear method and develop a standard operating procedure to be used in clinical trials that allows volume measurements in central lab. In order to satisfy that requirement, the effect of intermediate storage of the wicking spear in glass sample collection tubes sealed with a substantially waterproof and airtight stopper after fluid collection and shipment from clinical site to central lab was investigated.

### Materials

Capillary tubes—reference—(Radiometer, Capillary Tube-Denmark). Wicking spear, catalogue number 6040415 (Ultracell Medical Technology—US). Glass microscope slides. Eppendorf pipettors 0 to 10 μL and 10 to 100 μL with corresponding pipetors tips. Vacutainer™ sample collection tube catalogue number 367525 (BD Medical—Preanalytical Solution, US). Balance capable of tenths of a milligram. 0.09% NaCl solution for parenteral injection (Abbott, US, Laboratoire Aguettant, France).

### Example 1

The first example demonstrates the evaluation of the fluid recovery percentage. Fifteen (15) samples were tested using each collection method, at each of three different baseline dispensed fluid set points (2, 10 and 25 μL). The dispensed fluid volumes were placed onto microscope slides using Eppendorf pipettors, intended to simulate a known volume of saline leakage to be collected. The dispensed volume recorded weight was used as the baseline “known” value for collection volume delta and percentage recovery calculation. In the case of 25 μL dispensed set point, three capillary tubes were required to collect this fluid volume; therefore a total fluid column height/length measurement in mm, combining all three individual tube measurements. In contrast, only one wicking spear was required for collecting 25 μL. In order to normalize out variability in baseline dispensed volume between samples or groups, collection loss or volume delta (difference between as-calculated collected volume and baseline dispensed volume) and method accuracy or percentage recovery (collected volume as a percentage of baseline dispensed volume) were calculated and those used to evaluate and compare test method capability.

### Example 2

The second example demonstrates an evaluation of the operator effect on fluid collection method. The sample size was again fifteen (15) tests for each leakage collection method. However, only two different baselines dispensed fluid volume set points (2 and 25 μL) were tested with two different operators. One operator performed all dispensing of baseline leakage volumes onto the microscope slides to prevent introducing additional variability at this baseline starting point. Each test method was carried out by both test/measurement operators per the procedure described above.

### Example 3

The third Example demonstrates development and validation of fluid leakage measurement kit 10. At day 0, one operator prepared 122 fluid collection devices 20 stored in individual collection tubes 40. At day 0, Fifty-Six (56) tubes 40 (each sealed with caps 20) were used to investigate test method variability, reproducibility and repeatability, ten the tubes 40 were stored at room temperature for 12 days. Three operators were involved in test tubes weight measurement, adjusting balance zero at each measurement. Each measure was done in triplicate. The operator reproducibility based evaluate from 10 subsequent measurements of tubes selected in random fashion. An additional fifty-six (56) tubes were prepared by the same operator to evaluate the impact of storage temperature +4° C. for a period of 12 days. The one-hundred-twenty-two (122) tubes were weighed again by three operators at day three, day six, day nine and day twelve. The fluid leakage measurement variability was analyzed by ANOVA according to dispensed fluid volumes 0, 2, 4, 8, 16, 32, 64 μL, to the operator performing fluid collection and the weighting of tubes containing the fluid collection device 30 before and after dispensed fluid collection, the collection tube storage after fluid collection form day 0 to day 12. The method reproducibility was represented by the variability σ² operator, σ² dispensed volume*operator and σ² collected volume*operator.

The statistical analysis for all the preceding examples was conducted with Minitab 2-sample T-tests with a confidence interval setting of 95%. P-values of 0.05 or less are considered statistically significant. The 2-sample T-tests with confidence intervals were carried out on collected fluid, collection loss or volume delta by test method, and percentage recovery by test method at each dispensed volumes. Boxplots were generated for percentage recovery by test method at each of the three dispensed volume. ANOVA General Linear Model with multiple comparison confidence intervals were run at both 2 μL and 25 μL leakage volume set points to assess method performance versus acceptance criteria as well as method-to-method and operator to operator difference looking for statistical significance. The same ANOVA method was used to evaluate time and temperature effect on collected wicking spears stored in Vacutainer collection tubes.

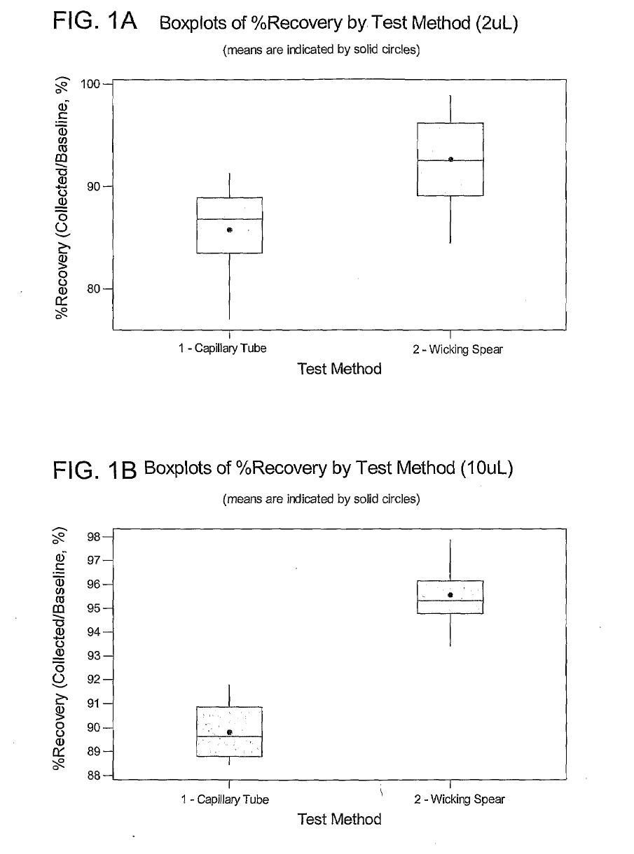

A shown in Table 1, first data column, the baseline initial dispensed volume on the microscope slides for both test method groups, at each volume set point are not significantly different. The wicking spear method has significantly lower leakage volume collection loss than the capillary tube method. Similarly, the wicking spear method collects significantly more fluid volume than the capillary tube method, regardless of initial leakage amount. FIG. 1A to 1C indicate that the wicking spear collection method has a significantly higher and better leakage collection percentage recovery of the initial dispensed volume than the capillary tube method. The 95% confidence interval range of percentage recovery improvement using the wicking spear method over all dispensed volume set points is between 4.52% and 7.08%. The significant lowest method accuracy or percentage recovery performance for both tests was observed with 2 μL set point. Evaporative loss may play a role as adverse impact notably on the percentage recovery.

TABLE 1
Comparison of the capability of the two fluid leakage collection methods

| | | | | | | Cap. vs. wick |
|---|---|---|---|---|---|---|
| | | | | | | 95% confidence |
| | | | | | | interval |
| | | | | 3(2/1) | | [Collection Loss |
| | | 1 | 2 | Method | 4 (1-2) | difference] |
| | | Dispensed | Collected | Accuracy | Collection | (% Recovery |
| Leakage collection | | Volume (μL) | Volume (μl) | % recovery | Loss (μL) | Difference)_{—} |
| method | | Mean and standard deviation | | | | |
| 2 μL | Capillary tube | 1.82 ± 0.054 | 1.56 ± 0.063 | 85.8 ± 4.09 | 0.26 ± 0.080 | [0.067 → 0.184] |
| | Wicking spear | 1.83 ± 0.069 | 1.70 ± 0.076 | 92.7 ± 4.07 | 0.14 ± 0.077 | (3.87% 9.95%) |
| | T-test (p value) | 0.641 | 0.000 | 0.000 | 0.000 | |
| 10 μL | Capillary tube | 9.85 ± 0.069 | 8.85 ± 0.113 | 89.9 ± 1.06 | 1.00 ± 0.105 | [0.486 → 0.647] |
| | Wicking spear | 9.80 ± 0.152 | 9.36 ± 0.183 | 95.6 ± 1.11 | 0.44 ± 0.109 | (4.91% 6.53%) |
| | T-test (p value) | 0.254 | 0.000 | 0.000 | 0.000 | |
| 25 μL | Capillary tube | 24.58 ± 0.196 | 22.35 ± 0.368 | 90.9 ± 0.93 | 2.23 ± 0.217 | [1.024 → 1.316] |
| | Wicking spear | 24.63 ± 0.115 | 23.56 ± 0.176 | 95.7 ± 0.68 | 1.06 ± 0.170 | (4.16% 5.38%) |
| | T-test (p value) | 0.409 | 0.000 | 0.000 | 0.000 | |

Table 2 indicates that both methods reach the acceptance criteria for method validation as evaluated by the average accuracy calculated by the percentage of recovery. At both 2 μL and 25 μL leakage volume set points. The analysis of variance within operator indicates an overall significant test method difference (p=0.034 and p<0.0005), operator difference (p=0.020 and p<0.005, as well as operator by method interaction difference (p=0.003 and p<0.0005) detected for both average accuracy (percentage of recovery) and average collection loss (volume delta) responses. When examining the 95% simultaneous confidence intervals, the overall operator significant difference result only from difference in the capillary tube method, and not the wicking spear method. Nevertheless, despite the incorporation of this operator difference, both methods were still able to exceed the minimum acceptance criteria.

TABLE 2
Test method validation based on acceptance criteria.

| | | Percentage recovery | Collection loss (μL) |
|---|---|---|---|
| | CI 95% | Lower bound | Upper bound |
| 2 μL | Capillary tube | 86.O% | 0.365 |
| | Wicking spear | 83.5% | 0.411 |
| 25 μL | Capillary tube | 91.6% | 1.84 |
| | Wicking spear | 95.2% | 0.956 |

Acceptance criteria:
At 2 μL, average accuracy (% recovery) of ≧70%
At 25 μL, average accuracy (% recovery) of ≧85%

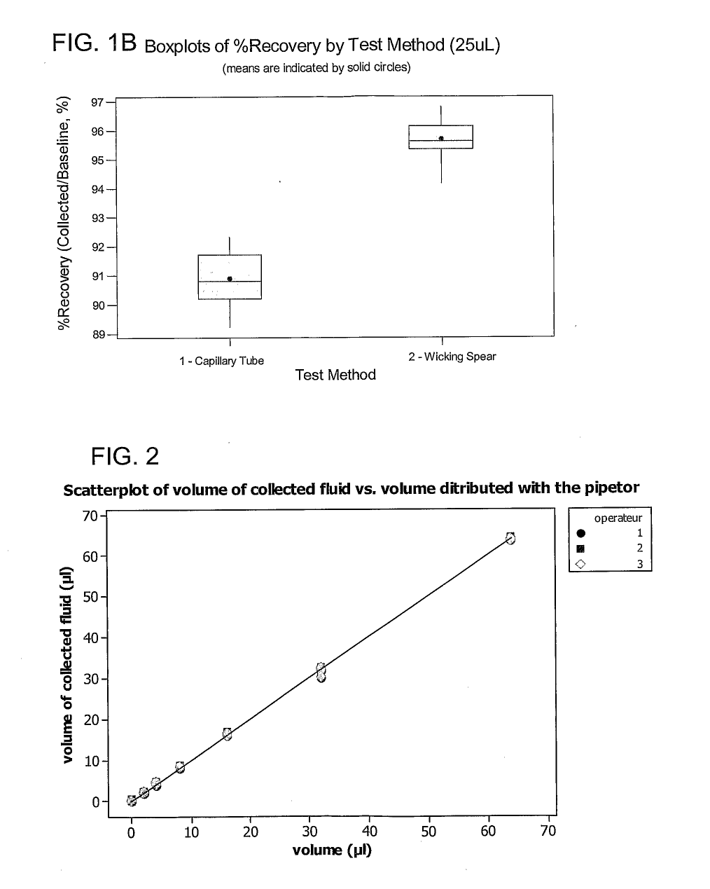

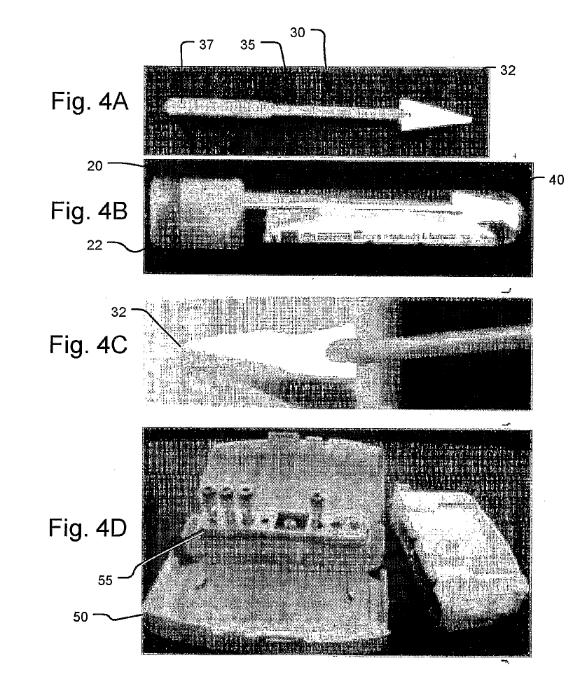

Based on fluid recovery percentage (fluid collection accuracy) and the consistency of the collection method according to the operator as well as the easier handling of the wicking spear, the wicking spear method has been selected as the most consistent to develop a fluid leakage measurement kit. The kit consists in a 7 ml glass, dry, Vacutainer™ Brand sample collection tube containing one wicking spear, which are weighted before and after fluid collection. For shipment the Vacutainer™ Brand collection tube containing a wicking spear are packaged a box for shipment of biological sample (BD Medical—Preanalytical Solution—US) as shown in FIG. 4B. FIG. 2 shows the linear interaction between the dispensed fluid volume on microscope slide and the collected volume in the wicking spear. The measurement of the delta volume (collected volume) using wicking spear packaged in Vacutainer™ Brand sample collection tube do not alter the measure accuracy. The table 3 shows that the method variability is constant whatever the dispensed volume.

TABLE 3
Variability of wicking spear method using collection
tube for storage according to the dispensed fluid.

| Dispensed volume | | | |
|---|---|---|---|
| (μL) | σ² Method | σ² Repeatability | σ² Reproducibility |
| 0 | 0.135 | 0.101 | 0.090 |
| 2 | 0.125 | 0.091 | 0.084 |
| 4 | 0.154 | 0.096 | 0.120 |
| 8 | 0.119 | 0.097 | 0.068 |
| 16 | 0.149 | 0.103 | 0.108 |
| 32 | 0.147 | 0.112 | 0.095 |
| 64 | 0.142 | 0.105 | 0.097 |

The ANOVA General Linear Model with multiple comparisons was performed at each dispensed volume set points (0 to 64 μL) to assess time and storage temperature effects looking at statistical significance. The two factors (time and temperature) as well as their interaction have a significant interaction on the measurement of the collected volume. After 9 and 12 days of storages the change in measured collected volume is always below 1 μL, which is considered as not clinically relevant. The storage at +4° C. did provide improvement in the of the measured volume stability over the period of time.

Aspects of the present invention provide a consistent device and method for collecting and measuring fluid leakage volumes at the injection site of drug delivery after hypodermic) intradermal or intramuscular injection. Such a device and method may be required for injection completeness and dose delivery accuracy evaluation in clinical development of a new injection device. The wicking spear gravimetric method of certain aspects of the invention provides for easier and more consistent method for fluid leakage collection. The fluid leakage collection method accuracy performance range for particular embodiments of the present invention are (±3σ) for 2 μL volume from 80.49% to 104.91%; for 10 μL from 92.27% to 98.93% and for 25 μl from 93.66% to 97.74%. The kit using Vacutainer™ glass sample collection tube to store the wicking spears before and after fluid leakage collection provides reliable equipment for fluid collection device intermediate storage and shipment to a central lab for weight/mass measurements. Weighing the kits in a central lab in a batch fashion before and after fluid contributes to minimize operator and balance errors on method accuracy. For certain embodiments of the invention, the volume detection threshold is below 2 μl and the measurement error, even after 12 days of storage at room temperature, is below 1 μl, which is consider as non clinically relevant.

We claim:

1. A method for the collection of a fluid emanating from an injection site, after an injection has been performed comprising:
providing a kit having:
a wicking element comprised of a porous material having a distal tip;
a container including a closure having at least one closable opening wherein said closure substantially seals said closable opening and wherein the container, the closure and a container/closure interface is impermeable to loss of said fluid or a constituent of said fluid for a period of time;
weighing at least said wicking element prior to use and determining a first mass;
recording the mass value;
collecting said fluid into said wicking element;
placing said wicking element within said container;
weighing at least said wicking element after use and determining a second mass; and,
subtracting said first mass from said second mass to determine a fluid leakage mass.

2. A method according to claim 1 wherein said first and second weighing steps include weighing said container and closure.

3. A method according to claim 1 further comprising calculating a fluid leakage volume wherein said fluid leakage volume is computed using said fluid leakage mass and a known density of the fluid.

4. A method according to claim 1 further comprising selecting a wick size of said wicking element wherein said wicking element has a dimension L and has a predetermined average pore diameter wherein the wick size is determined using a predetermined maximum collection volume of said fluid, a predetermined viscosity of said fluid and a predetermined surface tension of said fluid.

5. A method according to claim 4, wherein said selecting said wick size includes factoring a predetermined maximum collection time for said fluid into selecting said wick size.

6. A method according to claim 5 wherein selecting said wick size having said dimension L is determined using the formulae:
$${L}^{2} = \frac{γ Dt}{4 η}$$
wherein D is the predetermined average pore diameter of said wicking element and t is the predetermined maximum collection time for said fluid, and η is the predetermined viscosity of said fluid and γ is the predetermined surface tension of said fluid, and dimension L is correlated to the volume of said wicking element.

7. A kit for the collection of a fluid from an injection site, after an injection has been performed comprising:
an elongated body said body having a distal end and a proximal end and a wicking element comprised of a porous material having a dimension L wherein said porous element is located at said distal end of said body;
a substantially impermeable container having at least one closable opening wherein said container is sized to completely contain said elongated body and said wicking element, wherein the container is impermeable to loss of said fluid or a constituent of said fluid for a period of time; and
a closure wherein said closure substantially seals said closable opening.

8. A kit according to claim 7 wherein a wick size of said wicking element having a predetermined average pore diameter of said wicking element wherein the wick size is determined using a predetermined maximum collection volume of said fluid, a predetermined viscosity of said fluid and a predetermined surface tension of said fluid.

9. A kit according to claim 8, wherein said wick size is correlated to a predetermined maximum collection time for said fluid into selecting said wick size.

10. A kit according to claim 8 wherein said dimension L correlated is to said wick volume, and dimension L is calculated by
$$\sqrt{\frac{γ Dt}{4 η}}$$
wherein D is the predetermined average pore diameter of said wicking element and t is the predetermined maximum collection time for said fluid, and q is the predetermined viscosity of said fluid and γ is the predetermined surface tension of said fluid.
